(19) United States
(12) Patent Application Publication
(10) Pub. No.: US 2022/0054595 A1
(43) Pub. Date: Feb. 24, 2022

(54) Formulation Including a Combination of beta-Endorphin and Adrenocorticotropic Hormone

(71) Applicant: Adamis Pharmaceuticals Corporation, San Diego, CA (US)

(72) Inventors: Charles Peter Lollo, San Diego, CA (US); Dennis J. Carlo, San Diego, CA (US)

(21) Appl. No.: 17/438,829

(22) PCT Filed: Mar. 13, 2020
(86) PCT No.: PCT/US2020/022473

§ 371 Date: Sep. 13, 2021

Related U.S. Application Data
(60) Provisional application No. 62/818,034, filed on Mar. 13, 2019.

(51) Int. Cl. A61K 38/35; A61K 38/33; A61K 31/445; A61P 25/28

(52) U.S. Cl. CPC A61K 38/35; A61P 25/28; A61K 31/445; A61K 38/33

(57) ABSTRACT

The present specification provides compositions of β-endorphin and adrenocorticotropic hormone (ACTH). These compositions are useful in methods of treating disease.

### CROSS REFERENCE TO RELATED APPLICATIONS

This application claims the benefit of U.S. Provisional Patent Application Nos. 62/818,034, filed Mar. 13, 2019 and 62/981,379, filed Feb. 25, 2020, each of which is incorporated herein by reference in their entirety.

### FIELD

The present disclosure is directed to co-formulations of β-endorphin and adrenocorticotropic hormone (ACTH) as well as their use in methods of treating mammalian disease.

### BACKGROUND

Extracts from animal pituitary glands have been used since the 1950s to treat numerous disease indications including multiple sclerosis, systemic lupus erythematosus, proteinuria in nephrotic syndrome, polymyositis, symptomatic sarcoidosis, psoriatic arthritis, rheumatoid arthritis, ankylosing spondylitis, severe acute and chronic allergic and inflammatory processes involving the eye, and infantile spasms. These extract products contain hundreds of peptides that mostly derive from pro-opiomelanocortin (POMC). POMC is cleaved in vivo into several major peptides including adrenocorticotropic hormone (ACTH), melanocyte stimulating hormones (α, β, and γ), Lipotropin (β and γ), β-endorphin and Met-Enkephalin. In addition, peptide extracts contain hundreds of peptide forms that are generated by post-translational modifications.

The activity of such preparations has generally been attributed to ACTH and its ability to stimulate the adrenal cortex to secrete cortisol, corticosterone, and aldosterone. In more recent years synthetic analogues of ACTH have been marketed, such as tetracosactide or cosyntropin (e.g., Cortrosyn® by Amphastar; Synacthen® by Mallinckrodt), consisting of the first 24 amino acids of ACTH's 39 amino acids. While this truncated form of ACTH is reported to have the same corticosteroidogenic activity as natural ACTH, this compound has only been approved for diagnostic uses, for screening patients presumed to have adrenal insufficiency; therapeutic efficacy has not been recognized by the Food and Drug Administration. Cosyntropin has been tested as a treatment for infantile spasms but gave poorer outcomes than prednisolone. Moreover, manufacturers of the traditional biologics (which are defined more by the process by which they are made than what the contents of the product are) have suggested that therapeutic efficacy arises in part from additional unidentified components of the pituitary extracts. Indeed, pituitary extracts have effects that go beyond steroidogenesis. Thus, there has existed an unaddressed need for a defined composition reproducing the therapeutic efficacy of the traditional biologic preparation.

### SUMMARY

Disclosed herein are compositions, particularly pharmaceutical compositions, comprising β-endorphin and adrenocorticotropic hormone (ACTH) or analogues of one, the other, or both, thereof. In some embodiments the β-endorphin or ACTH have the human sequence of SEQ ID NO: 1 and SEQ ID NO: 2, respectively. In alternative embodiments the β-endorphin or ACTH, have the bovine sequence of SEQ ID NO: 3 and SEQ ID NO: 4, respectively, or the porcine sequence of SEQ ID NO: 5 and SEQ ID NO: 6 or 7, respectively. ACTH and β-endorphin are highly conserved and the native sequences from other species may be considered analogues of one or another of the sequences enumerated herein. In other embodiments, the analogue is a truncation of a natural sequence, for example, ACTH (1-24). Still other analogues incorporate one or more D-amino acids, for example, amino acids 1 to 5 of the N-terminal residues.

In some embodiments, compositions comprising β-endorphin ACTH or analogues thereof do not comprise any active peptides comprising amino acid sequences from other regions of POMC. In some embodiments, compositions comprising β-endorphin and ACTH or analogues thereof do not comprise any other active peptides derived from POMC. In some embodiments, compositions comprising β-endorphin and ACTH or analogues thereof do not comprise α-melanocyte-stimulating hormone (α-MSH), corticotropin-like intermediate lobe peptide (CLIP), β-lipotropin (β-LPH), μ-MSH, γ-MSH, or N-POMC, or any combination thereof.

Some compositions are pharmaceutical co-formulations of β-endorphin and ACTH. In some embodiments, the formulation is a repository or depot-forming formulation, for example, an injectable gel. In some embodiments the injectable gel comprises gelatin, for example, 16% gelatin.

In some embodiments, the co-formulation is supplied in a vial. In other embodiments, the co-formulation is supplied in a pre-filled syringe.

Disclosed herein are methods of treating a disease, disorder, or condition comprising administering a co-formulation of β-endorphin and ACTH, use of β-endorphin and ACTH for treating a disease, use of β-endorphin and ACTH in the manufacture of a medicament for treating a disease, and β-endorphin and ACTH for use in treating a disease. Some embodiments further comprise administration of a superoxide dismutase mimetic, for example Tempol (4-oxypiperidol).

In various embodiments, the disease to be treated is an autoimmune disease, an inflammatory disorder, or an allergic disorder. In some embodiments, the disease to be treated is multiple sclerosis, systemic lupus erythematosus, proteinuria in nephrotic syndrome, polymyositis, symptomatic sarcoidosis, psoriatic arthritis, rheumatoid arthritis, ankylosing spondylitis, or severe acute and chronic allergic or inflammatory processes involving the eye. In some embodiments, the disease to be treated is a neurologic or seizure disorder, including various types of childhood epilepsy, for example, infantile spasms (West syndrome), Lennox-Gastaut syndrome, Landau-Kleffner syndrome, and electrical status epilepticus in sleep.

### DETAILED DESCRIPTION

Disclosed herein are compositions and formulations comprising β-endorphin and adrenocorticotropic hormone (ACTH) or analogues of either or both thereof. β-endorphin and ACTH are both naturally derived by proteolytic processing from proopiomelanocortin (POMC).

Human POMC is a 241 amino acid polypeptide (SEQ ID NO: 10) which gives rise to multiple recognized peptides. Residues 1-26 are a signal peptide; residues 27-102 are NPP; residues 77-87 are γ-melanocyte-stimulating hormone (γ-MSH); residues 105-134 are a “potential peptide”; residues 138-176 are ACTH, also called corticotropin; residues 138-150 are α-MSH; residues 156-176 are corticotropin-like intermediate lobe peptide (CLIP); residues 179-267 are β-lipotropin (β-LPH); residues 179-234 are γ-LPH; residues 217-234 are β-MSH; residues 237-267 are β-endorphin; and residues 237-241 are met-enkephalin; as described at UniProtKB—P01189 (COLI_HUMAN).

The sequence of human POMC is:

(SEQ ID NO: 10)
MPRSCCSRSGALLLALLLQASMEVRGWCLSSQCQDLTTESNLLECIRACK
PDLSAETPMFPGNGDEQPLTENPRKYVMGHFRWDRFGRRNSSSSGSSGAG
QKREDVSAGEDCGPLPEGGPEPRSDGAKPGPREGKRSYSMEHFRWGKPVG
KKRRPVKVYPNGAEDESAEAFPLEFKRELTGQRLREGDGPDGPADDGAGA
QADLEHSLLVAAEKKDEGPYRMEHFRWGSPPKDKRYGGFMTSEKSQTPLV
TLFKNAIIKNAYKKGE.

In another common numbering system, the N-terminal amino acid residue of ACTH is considered 1, so that ACTH is amino acid residues 1-39, α-MSH is 1-13, CLIP is 19-39, β-LPH is 42-130, γ-LPH is 42-97, β-MSH is 80-97, β-endorphin is 100-130, Met-enkephalin is 100-104, γ-endorphin is 100-116, and α-endorphin is 100-115, of SEQ ID NO:11. Thus the sequence of SEQ ID NO: 11 is:

SYSMEHFRWGKPVGKKRRPVKVYPNGAEDESAEAFPLEFKRELTGQRLRE
GDGPDGPADDGAGAQADLEHSLLVAAEKKDEGPYRMEHFRWGSPPKDKRY
GGFMTSEKSQTPLVTLFKNAIIKNAYKKGE.

β-endorphin is a 31 amino acid peptide. The sequences of human, bovine, porcine, and murine β-endorphin peptides are each presented in Table 1.

TABLE 1
β-endorphin sequences

| | | | SEQ |
|---|---|---|---|
| Species | Accession* | Sequence** | ID NO. |
| Human | 764134A | YGGFMTSEKS QTPLVTLFKN | 1 |
| | | AIIKNAYKKG E | |
| Bovine | NP_776576 | YGGFMTSEKS QTPLVTLFKN | 3 |
| | | AIIKNAHKKG | |
| Porcine | P01192 | YGGFMTSEKS QTPLVTLFKN | 5 |
| | | AIVKNAHKKG | |
| Murine | NP_032921 | YGGFMTSEKS QTPLVTLFKN | 8 |
| | | AIIKNAHKKG | |

*NCBI Protein Database Accession number
**Differences from the human sequence are bold and underlined

The sequences of β-endorphins from many other species have been determined and are readily available from public protein sequence databases and are to be considered analogues of the above disclosed peptides to the extent that they differ. Various embodiments of the herein disclosed compositions and formulations may specifically include one or more of the above peptides (in combination or as alternatives), or an analogue thereof. Other embodiments of the herein disclosed compositions and formulations specifically exclude one or more of the above peptides, or analogues thereof.

β-endorphin is concentrated in the anterior pituitary and pars intermedia, but also widely distributed throughout the body and has been detected in nervous tissue as well as adrenals, heart, liver, and placenta. Concomitant with its broad distribution, the physiological activity of β-endorphin directly or indirectly includes neurotransmitter functions, suppressive effects on respiration, analgesia, maintenance of cardiovascular homeostasis, and even regulation of T- and B-cell function in the immune system. β-endorphin's activity is mediated through classical μ, δ and κ opioid receptors and perhaps a more selective epsilon receptor. β-endorphin, like Met-enkephalin, contains the amino acid sequence YGG (residues 1-3) and binds to opioid receptors (μ, δ, and κ) which mediate its physiologic functions. However, the signaling pathways associated with β-endorphin's roles in the pathophysiology of pain management, obesity, and even immunological responsiveness have not been fully described. In some embodiments, β-endorphin analogues comprise the YGG subsequence.

Other analyses of β-endorphin reveal that YGG (residues 1-3) and QTPLV (residues 11-15) are important for binding to the human β opioid receptor; K9, QTPLV (residues 11-15), FK (residues 18-19), and K29 are important for binding to the human δ opioid receptor; and YGGF (residues 1-4), T6, K9, F18, I22, A26, Y27, K29 and E31 are important for binding to the human κ opioid receptor. (Individual conserved residues are denoted simply as the amino acid and the residue position, so that K9 indicates the lysine that is the ninth residue.) It is noted that Y27 and E31 are not conserved across species; apparently these substitutions can be tolerated, or the activities of the non-human β-endorphins in human systems are not crucially dependent on binding to the human κ opioid receptor. In various embodiments, β-endorphin analogues comprise one, some, or all of these conserved subsequences or individual amino acid residues.

ACTH is a 39 amino acid peptide. The sequences of human, bovine, porcine, and murine ACTH peptides are each presented in Table 2.

TABLE 2
ACTH sequences

| | | | SEQ |
|---|---|---|---|
| Species | Accession* | Sequence** | ID NO. |
| Human | P01189 | SYSMEHFRWGKPVGKKRRPVK | 2 |
| | | VYPNGAEDESAEAFPLEF | |
| Bovine | AAB59262 | SYSMEHFRWGKPVGKKRRPVK | 4 |
| | | VYPNGAEDESAQAFPLEF | |
| Porcine | NP_999023 | SYSMEHFRWGKPVGKKRRPVK | 6 |
| | | VYPNGAEDELAEAFPLEF | |
| Porcine | 560078A^{†} | SYSMEHFRWGKPVGKKRRPVK | 7 |
| | | VYPDGAEDQLAEAFPLEF | |
| Murine | AAA37169 | SYSMEHFRWGKPVGKKRRPVK | 9 |
| | | VYPNVAENESAEAFPLEF | |

*NCBI Protein Database Accession number
**Differences from the human sequence are bold and underlined
^{†}as found in H.P. ACTHAR ® Gel

The sequences of ACTH from many other species have been determined and are readily available from public protein sequence databases and are to be considered analogues of the above disclosed peptides to the extent that they differ. Various embodiments of the herein disclosed compositions and formulations may specifically include one or more of the above peptides that can be derived from POMC (in combination or as alternatives), or an analogue thereof. Other embodiments of the herein disclosed compositions and formulations specifically exclude one or more of the above peptides that can be derived from POMC, or analogues thereof. In some embodiments, the disclosed compositions are substantially free of other peptides that can be derived from POMC. In various embodiments, ACTH and β-endorphin, or analogues thereof, together make greater than 90%, 95%, 98%, 99%, 99.5%, 99.8%, 99.9% or 99.99% w/w of the peptides that can be derived from POMC that are present in a disclosed composition or formulation. In various embodiments, ACTH and β-endorphin, or analogues thereof, together make greater than 90%, 95%, 98%, 99%, 99.5%, 99.8%, 99.9% or 99.99%, on a molar basis, of the peptides that can be derived from POMC that are present in a disclosed composition or formulation.

ACTH acts through the melanocortin receptor 2 (MC2R) which is mainly found in the zona fasiculata of the adrenal cortex. By binding to this receptor, ACTH stimulates production of glucocorticoids by adrenocortical cells. This binding is dependent on the KKRRP sequence (residues 15-19) in ACTH, which is not found in other peptides derived from POMC. MC2R is also found on white and brown adipocytes, consistent with ACTH's observed role in glucose metabolism on osteoblasts, and in the skin. ACTH also contains the sequence HFRW (residues 6-9) which mediates binding to all five of the melanocortin receptors. Collectively, the five melanocortin receptors are involved in a diverse number of physiological functions, including pigmentation, steroidogenesis, energy homeostasis, exocrine secretion, sexual function, analgesia and inflammation. Thus, it is clear that ACTH can have functions beyond steroidogenesis. In some embodiments ACTH analogues comprise the KKRRP subsequence, the HFRW subsequence, or both.

The ACTH and β-endorphin used to make the herein disclosed compositions and formulations can be obtained by any of several methods. Both of these peptides are small enough that it is commercially feasible to produce them by chemical synthesis using standard techniques. Chemical synthesis offers the advantages of using the human amino acid sequences, avoids exposure to enzymes that might further cleave the desired peptide, and obviates fears about adventitious infectious agents (be they prions, viruses, or bacteria) that can attend biologically sourced material.

Alternatively, they can be purified from pituitary extracts similar to those used in the manufacture of historic and currently marketed ACTH-based products. However, a more extensive purification can be used to obtain each of the peptides in (near) homogenous form. In some embodiments, affinity chromatography can be used. Anti-ACTH and anti-β-endorphin monoclonal antibodies have been successfully made in the past and some are commercially available as laboratory reagents. Thus in some embodiments, the composition or formulation comprising ACTH and β-endorphin is substantially free of other peptides found in pituitary extract. In various embodiments, ACTH and β-endorphin, or analogues thereof, in a disclosed composition or formulation are greater than 90%, 95%, 98%, 99%, 99.5%, 99.8%, 99.9% or 99.99% (w/w) free of other peptides found in pituitary extract. In various embodiments, ACTH and β-endorphin, or analogues thereof, in a disclosed composition or formulation are greater than 90%, 95%, 98%, 99%, 99.5%, 99.8%, 99.9% or 99.99% free of other peptides found in pituitary extract on a molar basis.

ACTH and β-endorphin can also be made using recombinant DNA technology either individually or from a common precursor. Post-translational modification of the final peptide is not necessary, so they may be produced in bacterial or eukaryotic culture systems. The primary translation product can typically have an initiator methionine, which can either be retained or be part of a sequence that can be subsequently cleaved.

As used herein the term “analogue” can refer to any variant that retains sufficient structure and relevant activity to be recognized as being related to the base peptide and to serve a similar function. In some embodiments an analogue contains one or more amino acid substitutions, insertions or deletions. In some embodiments an analogue is a truncated version of the base peptide. In some embodiments additional amino acid sequences have been added to one or the other termini of the base peptide. Such additional sequences may aid in the purification of the peptide, or alter its bioavailability or half-life after administration. In some embodiments the analogue can include non-amino acid additions to the peptide, for example, PEGylation.

The herein disclosed compositions, including pharmaceutical compositions, and formulations may contain water or other solvents, salts, buffers, stabilizers, anti-bacterial or anti-fungal agents, or other excipients as are used in pharmaceutical products. Excipients can include: phenol, for example, 0.5%; added cysteine <1.1%; NaOH or acetic acid to adjust pH; and water for injection.

As used herein the term “pharmaceutical composition” refers to a composition that is suitable for use as a drug product, including that it facially meets safety standards as would be required by regulatory authorities such as the Food and Drug Administration or corresponding agencies outside the U.S. This is not meant to necessarily imply any particular level of laboratory or clinical testing to demonstrate safety. Rather it refers to meeting appropriate standards of sterility or being aseptic, and excludes substances that would be considered unacceptable due to the harm or potential harm they could cause the patient (human or veterinary, as appropriate), or the active agents; such exclusion being absolute or based on not exceeding an acceptable concentration or amount. In some embodiments, it can also refer to comprising sufficient quantity of active agent to be expected to produce a physiologic effect.

Some embodiments are repository (or depot) formulations of β-endorphin and ACTH. Such formulations release the peptides over time, prolonging their effective half-life and reducing the number of administrations needed in a course of treatment or other time interval. In some embodiments, the repository formulation contains gelatin, for example 13-19% gelatin, for example 16% gelatin, or any sub-range or individual value therein obtained by steps of 0.5%. Alternatively, a copolymer such as poly(lactic-co-glycolic acid) (PLGA) could be used instead of gelatin. A similar half-life prolonging effect can be obtained by PEGylation of the peptide. In some embodiments polyethylene glycol (PEG) is covalently attached to a terminal C residue added to the sequence of the peptide.

In some embodiments, the formulation is provided in a vial. In other embodiments the formulation is provided in a pre-filled syringe. In some embodiments the formulation contains 80 USP units of ACTH per mL. In some embodiments a dose contains approximately half the molar amount of β-endorphin as ACTH. In another embodiment, the formulation contains 0.5 to 2 mg/mL of ACTH and 0.1 to 1 mg/mL of β-endorphin.

For most uses, a dosage comprising 40-80 U of ACTH in a repository (or other extended half-life) formulation over a 24-72 hour period is appropriate. The dosage can be administered by subcutaneous or intramuscular injection and can be administered by a healthcare professional (for example, a doctor, a nurse, a physician's assistant, or the like) or by a trained patient, a trained parent of a patient, or other trained non-professional assistant. In some embodiments administration is viewed as taking place under the direction of and at the behest of the prescribing physician. In other embodiments administration is viewed as taking place at the behest of the recipient patient. In all cases administration should be viewed as being carried out in order to receive the benefits attendant to the treatment.

The two peptides, β-endorphin and ACTH, bind to all of the melanocortin and opioid receptors bound by any of the peptides derived from POMC, and thus can be used to replicate the activity profile of pituitary extracts. In various embodiments, the herein disclosed compositions and formulations of β-endorphin and ACTH can be used to treat a variety of diseases and disorders currently treated with an ACTH-containing pituitary extract, as described in subsequent paragraphs. A combination of β-endorphin and ACTH can be equally or more effective than the pituitary extracts and provide a more completely defined medicament.

Children with various types of childhood epilepsy that may not respond to the usual seizure medicines can be candidates for treatment. Pituitary extracts comprising ACTH have been used primarily in the treatment of infantile spasm (West syndrome) but have also found to be useful in Lennox-Gastaut syndrome, Landau-Kleffner syndrome, and electrical status epilepticus in sleep. Improvement in seizure control has been seen even in the most difficult-to-control epilepsy after treatment with pituitary extract comprising ACTH, Treatment can also improve the child's developmental status.

It is unclear just how ACTH works to stop seizures. It may work directly on the brain in addition to stimulating the adrenal glands. There is some controversy about whether to use low-dose or high-dose ACTH when treating childhood seizures. No definitive study has yet established one dose as superior. However, a typical dosage would comprise 150 U of ACTH/m²/clay, divided into two daily intramuscular injections for two weeks. The dosage is then tapered down over a period of several (for example, 4) weeks. Additionally, patients with infantile spasms exhibit depressed levels of both ACTH and β-endorphin.

Acute exacerbations of multiple sclerosis can be treated. Treatment speeds resolution of the acute exacerbation, but has not been shown to impact the long-term course of the disease. However, the non-steroidogenic immune-modulating activities of ACTH support potential usefulness for long-term treatment of multiple sclerosis. Thus, the present compositions and formulations of β-endorphin and ACTH can be useful for chronic treatment of multiple sclerosis in addition to treating acute exacerbations.

Acute episodes or exacerbations of rheumatic disorders can be treated, such as psoriatic arthritis, rheumatoid arthritis, including juvenile rheumatoid arthritis, and ankylosing spondylitis. Typically, treatment constitutes short-term adjuvant therapy, but in some cases low dose maintenance therapy can be undertaken.

Collagen disease, such as systemic lupus erythematosus and systemic dermatomyositis (polymyositis) can be treated either to address exacerbations or as maintenance therapy.

In the realm of ophthalmic disease, severe acute and chronic allergic and inflammatory processes involving the eye and its adnexa (for example eyelids, lacrimal glands, and tear ducts) can be treated. Specific conditions treated can include keratitis, iritis, iridocyclitis, diffuse posterior uveitis and choroiditis, optic neuritis, chorioretinitis, and anterior segment inflammation.

In the realm of edema, the herein disclosed compositions and formulations can be used to induce diuresis or remission of proteinuria in the nephrotic syndrome without uremia of the idiopathic type or that due to lupus erythematosus.

The herein disclosed compositions and formulations can also be used to treat dermatologic diseases, such as severe erythema multiforme and Stevens-Johnson syndrome; allergic conditions, such as serum sickness; and respiratory disease, such as symptomatic sarcoidosis.

Thus, some embodiments are methods of treatment in which a composition or formulation comprising ACTH and β-endorphin, or analogues thereof, such as described herein above, are administered to a patient in need thereof. In various embodiments the patient in need thereof has an autoimmune disease, an inflammatory disorder, or an allergic disorder. In some embodiments, the disease to be treated is multiple sclerosis, systemic lupus erythematosus, proteinuria in nephrotic syndrome, polymyositis, symptomatic sarcoidosis, psoriatic arthritis, rheumatoid arthritis, ankylosing spondylitis, or severe acute and chronic allergic or inflammatory processes involving the eye. In some embodiments, the disease to be treated is a neurologic or seizure disorder, including venous types of childhood epilepsy, for example, infantile spasms (West syndrome), Lennox-Gastaut syndrome, Landau-Kleffner syndrome, and electrical status epilepticus in sleep. Corresponding embodiments include use of a composition or formulation comprising ACTH and β-endorphin, or analogues thereof, in the treatment of anyone of these diseases or conditions, and use of ACTH and β-endorphin, or analogues thereof, in the manufacture of a medicament for the treatment of anyone of these diseases or conditions. In some embodiments, the composition or formulation comprising ACTH and β-endorphin, or analogues thereof, is administered intramuscularly or subcutaneously. In various embodiments, the composition or formulation comprising ACTH and β-endorphin, or analogues thereof, is administered twice daily, daily, or every 2nd day.

In some embodiments the patient in need thereof is additionally treated with a superoxide dismutase mimetic. In some embodiments the superoxide dismutase mimetic is Tempol (4-oxypiperidol; 1-A-oxidanyl-2,2,6,6-tetramethylpiperidin-4-ol). In some embodiments, Tempol is in a separate composition than the ACTH and β-endorphin, or analogues thereof. This allows the Tempol to be administered according to a different schedule and/or different route of administration. In some embodiments Tempol is administered orally. In some embodiments, Tempol is administered topically, sublingually, transrectally, intramuscularly, intravenously, or subcutaneously. In some embodiments the dosage of Tempol is 0.01-1000 mg per day. In some embodiments Tempol is included in the composition or formulation comprising ACTH and β-endorphin, or analogues thereof.

### EXAMPLES

The following non-limiting examples are provided for illustrative purposes only in order to facilitate a more complete understanding of representative embodiments now contemplated. These examples should not be construed to limit any of the embodiments described in the present specification, including those pertaining to the methods of treating any of the above disorders.

### Example 1

### Effect of β-Endorphin and ACTH in an Animal Model of Infantile Spasms

The Ts65Dn mouse was initially developed as a model of Down's syndrome, but shows spontaneous spike-and-wave discharges (Cortez, M. A., et al., Pediatric Research 65(5):499-503 (2009). Gamma-aminobutyric acid B (GABA_{B}R) agonists, such as baclofen and γ-butyrolactone, induce acute epileptic extensor spasms (AEES) associated with epileptiform polyspike bursts and an electrodecremental response on the EEG. The GABA_{B}R agonist-treated Ts65Dn mouse shows the unique clinical, electrographic, and pharmacologic signature of infantile spasms and represents a valid, acute model of this disorder. Rodent (but not porcine) ACTH (SEQ ID NO: 9) can significantly reduce AEES.

Cohorts of TS65Dn mice are administered a GABA_{B}R agonist and treated with a vehicle control, murine ACTH₁₋₂₄, murine ACTH₁₋₃₉, murine β-endorphin (SEQ ID NO: 8), murine ACTH₁₋₂₄ plus β-endorphin, or murine ACTH₁₋₃₉ plus murine β-endorphin and evaluated for the reduction in the number of AEES and of electrodecremental response.

### Example 2

### Effect of β-Endorphin and ACTH in a Murine Model of Relapsing-Remitting Multiple Sclerosis

Experimental autoimmune encephalitis (EAE) is the standard model used to test potential therapies for multiple sclerosis. One particular presentation of EAE is the SJL/J mouse sensitized with synthetic myelin basic protein 139-151 peptide (PLP₁₃₉₋₁₅₁), which induces a relapsing-remitting form of the disease (RR-EAE). H.P. Acthar Gel® (Acthar) has been shown to reduce mean clinical score during relapse and cumulative disease burden in comparison to placebo as well as ameliorating inflammation/demyelination in the spinal cord. Acthar treatment also suppresses ex vivo myelin peptide induced CD4⁺ T cell proliferation (Cusick et al., Autoimmunity 48(4):222-230 (2015).

RR-EAE is induced according to standard protocol. Briefly, SJL/J mice are sensitized by subcutaneous injection of with PLP₁₃₉₋₁₅₁ in complete Freund's adjuvant (CFA), followed by intravenous injections of Bordetella pertussis toxin. The mice are then randomized into the following treatment arms using a repository formulation: vehicle control, ACTH, β-endorphin, and ACTH plus β-endorphin; with comparison of human sequence to either porcine or murine sequence. In separate studies treatment is begun immediately upon relapse and five days after the onset of relapse. The mice are monitored daily for weight and clinical signs. Clinical scoring uses the scale: 0, no clinical signs; 1, loss of tail tonicity; 2, presents with mild hind leg paralysis with no obvious gait disturbance; 3, mild leg paralysis with gait disturbance and paralysis; 4, hind legs are paralyzed; and 5, moribund or dead. Histology is performed on spinal cords harvested after death by euthanasia and scored for inflammation and demyelination.

ACTH plus β-endorphin dampens RR-EAE and ameliorates inflammation and demyelination to a greater extent that ACTH alone.

### Example 3

### Effect of β-Endorphin and ACTH in EAE, a Murine Model of Relapsing-Remitting Multiple Sclerosis

The effect of administration of two dosages of ACTH and β-endorphin on severity and relapse in EAE was evaluated. EAE was induced in SJL mice by PLP₁₃₉₋₁₅₁/CFA immunization on Day 0. Treatment was late therapeutic, starting on Day 20 after immunization for each mouse. Mice were observed through the end of the study, Day 42 after immunization. Four groups of 20 mice each received one of the following treatments: vehicle (a negative control); 10 mg/kg oral prednisolone daily (reference treatment); 32 μg ACTH+16 μg endorphin by subcutaneous injection, every 2nd day (low dose); or 64 μg ACTH+32 μg β-endorphin by subcutaneous injection, every 2nd day (high dose).

EAE Induction

Mice were injected subcutaneously at four sites in the back with emulsion containing PLP₁₃₉₋₁₅₁ (native sequence) from HOOKE KIT™ PLP₁₃₉₋₁₅₁/CFA Emulsion, catalog number EK-0120 (Hooke Laboratories, Lawrence Mass.). Two sites of injection were in the upper back approximately 1 cm caudal of the neck line. The other two sites were in the lower back approximately 2 cm cranial of the base of the tail. The injection volume was 0.05 mL at each site. Even without treatment, most mice recover spontaneously after the first wave of disease. In the SJL model, EAE develops 10-15 days after immunization in 90-100% of immunized mice. The first wave of EAE lasts several days and most mice fully or almost fully recover from this first wave. After a disease-free period, which can last from one day to several months, most mice relapse. During the first 5-7 weeks after immunization, 50-100% of mice will develop a relapse. Each wave of paralysis results in body weight loss at onset, most of which is regained at recovery from the wave. The greatest weight loss is during the first wave of EAE. During the chronic phase of disease, the average EAE body weight is relatively stable, with normal slow increase with age. Since individual mice experience relapses at different times, only a few mice in each group are acutely sick at any given time after the first wave of EAE. Therefore, significant differences in relative body weight between groups are often not found after the first wave of disease in this model. In the vehicle treated group, disease developed as expected for this model. Five (5) mice were euthanized on Day 30 for analysis. Relapse occurred in 7 out of the 15 remaining mice, which were followed to Day 42, with typical course and severity of disease.

Treatment

100 EAE induced mice were initially considered a single group and were scored daily starting on Day 9 after immunization. On Day 20, mice were assigned to the experimental groups in a balanced manner to achieve groups with similar times of EAE onset, similar maximum EAE score and similar scores at the time of enrollment into treatment (see Table 2). The remaining mice, which developed EAE last, or which developed unusual signs of EAE such as head tilting, were not assigned to any treatment group.

TABLE 2
Treatment regimen

| | # of | | | | | | |
|---|---|---|---|---|---|---|---|
| Group | Animals | Treatment | Dose | Route | Frequency | Volume | Purpose |
| 1 | 20 | Vehicle | — | s.c. | Q2D | 0.1 mL | Negative |
| | | | | | | | control |
| 2 | 20 | Low Dose | ACTH: 32 μg | s.c. | Q2D | 0.1 mL | Test |
| | | | β-end: 16 μg | | | | |
| 3 | 20 | High Dose | ACTH: 64 μg | s.c. | Q2D | 0.1 mL | Test |
| | | | B-end: 32 μg | | | | |
| 4 | 20 | Prednisolone | 10 mg/kg | p.o. | QD | 10 mL/kg | Reference |

Vehicle was 16% gelatin from porcine skin (Sigma G2500-100G) prepared by dissolution in 60° C. sterile water with pH adjusted with 1 N NaOH to be 6.3 to 6.6, For the high dose treatment, porcine ACTH and porcine β-endorphin were dissolved in vehicle by heating to 80-90° C. for 20 minutes and then cooling to 60-70° C. followed by addition of ACTH and β-endorphin powder to achieve a concentration of 0.64 and 0.32 mg/ml solution, respectively, and stirring at 50-70° C. for 20 minutes; pH was between 6 and 7. For the low dose treatment this solution was diluted 1:2 with vehicle.

Treatment started on the day of group assignment (Day 20 after immunization). This was the time of the expected end of the first wave of EAE and before relapses were expected to begin. Treatment continued through Day 41. EAE score was determined daily by a person unaware of both treatment and of previous scores for each mouse. Weight was measured 3 times a week. Five mice in each group were euthanized on day 30 and the fifteen remaining mice euthanized at the end of the study (Day 42). Histological and immunohistological analyses were performed on tissue collected from the euthanized mice.

TABLE 3
EAE scoring criteria

| Score | Clinical observations |
|---|---|
| 0 | No obvious changes in motor functions of the mouse in comparison to non-immunized mice. |
| | When picked up by the tail, the tail has tension and is erect. Hind legs are usually spread apart. |
| | When the mouse is walking, there is no gait or head tilting. |
| 1 | Limp tail. When the mouse is picked up by the tail, instead of being erect, the whole tail drapes |
| | over your finger. |
| 2 | Limp tail and weakness of hind legs. When mouse is picked up by tail, legs are not spread apart, |
| | but held closer together. When the mouse is observed when walking, it has a clearly apparent |
| | wobbly walk. |
| 3 | Limp tail and complete paralysis of hind legs (most common). |
| | OR |
| | Limp tail with paralysis of one front and one hind leg. |
| | OR |
| | ALL of: |
| | Severe head tilting, |
| | Walking only along the edges of the cage, |
| | Pushing against the cage wall, |
| | Spinning when picked up by the tail. |
| 4 | Limp tad, complete hind leg and partial front leg paralysis. Mouse is minimally moving around the |
| | cage but appears alert and feeding. Usually, euthanasia is recommended after the mouse scores |
| | level 4 for 2 days. When the mouse is euthanized because of severe paralysis, score of 5 is |
| | entered for that mouse for the rest of the experiment. |
| 5 | Complete hind and complete front leg paralysis, no movement around the cage. OR Mouse is |
| | spontaneously rolling in the cage. OR Mouse is found dead due to paralysis. |

In-between scores were assigned when the clinical signs fell between defined scores.

Statistical analyses of the clinical findings are presented in Table 4 and of the histological and immunohistological finding are presented in Table 5. Overall disease severity in this experiment, as reflected in the mean maximal score (MMS), was moderate, limiting the statistical power of clinical observations. Nonetheless the MMS for high dose treatment was borderline statistically significant. Weight gain is another signal of clinical improvement. Both the low and high dose groups exhibited weight gain versus the vehicle control, with the high dose group showing clear statistical significance. In contrast, the prednisolone treatment group exhibited a statistically significant loss of weight versus the vehicle control emphasizing the marginal value of this treatment and indicating that even the low dose treatment with ACTH and β-endorphin provided a real improvement. Additionally, there was a trend toward reduced incidence of relapse in both the high and low dose groups that was greater than for the prednisolone treatment.

The histological analyses revealed a statistically significant reduction in inflammation and inflammatory infiltrates by both the low and high dose treatments, as evidenced by a reduction in the number of inflammatory foci as compared to the vehicle control. Similarly, both the low and high dose treatments led to a statistically significant reduction in demyelination, as evidenced by anti-myelin basic protein staining score. (Demyelination score: 0—<5% demyelinated area, 1—5-20% demyelinated area, 2—21-40% demyelinated area, 3—41-60% demyelinated area, 4—61-80% demyelinated area, and 5—81-100% demyelinated area,) Microglial activation, another indicator of inflammation, quantitated as the area of anti-lba1 staining, and gliosis (scarring), quantitated as the area of anti-GFAP staining were also both statistically significantly reduced, as compared to the vehicle control, by both the high and low dose treatment with ACTH and β-endorphin. In contrast, prednisolone treatment showed only at best a trend to reduction of inflammation and demyelination (and a higher level of gliosis), with none of these values approaching statistical significance. Collectively, these results indicate that treatment with ACTH and β-endorphin has greater efficacy in reducing EAE than prednisolone, especially at the higher of the two dosages tested, and support an expectation of similar beneficial effect in treating multiple sclerosis.

TABLE 4
Statistical analysis of clinical findings

| | | | | Mice not terminated on Day 30^{∥} | | | | | | | |
|---|---|---|---|---|---|---|---|---|---|---|---|
| | | | | | | MMS of | | | | | |
| | All mice | | | | | relapse | | | | | |
| | Mean | MMS of | | Incidence | | period ± SD | | | | | |
| | day of on- | first | Day 20 | of | p | (Days | | End | p | End % body | |
| Treatment | set ± SD | wave ± SD | score ± SD | relapse^{†} | value | 25-42)^{‡} | p value | score ± SD | value | weight ± SD | p value |
| Vehicle | 11.9 ± 0.8 | 3.30 ± 0.55 | 2.08 ± 0.59 | 46.7% | | 2.50 ± 0.87 | | 1.73 ± 1.02 | | 98.4% ± 6.8% | |
| Low Dose | 11.9 ± 0.7 | 3.25 ± 0.47 | 2.10 ± 0.53 | 20.0% | 0.1174 | 2.10 ± 0.57 | 0.2014 | 1.30 ± 0.86 | 0.1658 | 104.8% ± 11.0% | 0.0647 ** |
| High Dose | 12.0 ± 0.9 | 3.28 ± 0.53 | 2.05 ± 0.69 | 20.0% | 0.1174 | 1.77 ± 1.02 | 0.0502** | 1.10 ± 1.00 | 0.1060 | 112.5% ± 12.9% | 0.0009 * |
| Prednisolone, | 11.9 ± 0.7 | 3.33 ± 0.54 | 2.05 ± 0.65 | 33.3% | 0.4552 | 2.07 ± 1.10 | 0.2530 | 1.47 ± 1.04 | 0.3665 | 88.6% ± 9.3% | 0.0026 * |
| 10 mg/kg | | | | | | | | | | | |

* p < 0.05 vs. vehicle
** p < 0.10 vs. vehicle
^{†}Mice that relapsed as a proportion of surviving mice that developed the first wave of EAE
^{‡}Calculated for all mice in the group
^{∥}That is, mice followed until Day 42
Disease relapse compared using chi-square test
MMSs compared using Wilcoxon's non-parametric test
End EAE scores compared using Wilcoxon's non-parametric test
Change in body weight at the end of the study compared using two-tailed Student's t-test

TABLE 5
Statistical analysis of histological and immunohistological findings

| | Inflammatory | | Demyelination | | lba1 ± SD | | GFAP ± SD | |
|---|---|---|---|---|---|---|---|---|
| Treatment | foci/section ± SD | p value | (anti-MBP) ± SD | p value | (% of area) | p value | (% of area) | p value |
| Vehicle | 4.3 ± 2.8 | | 1.1 ± 0.6 | | 4.7 ± 1.6 | | 12.5 ± 1.8 | |
| Low Dose | 1.6 ± 1.0 | 0.0014* | 0.5 ± 0.4 | 0.0084* | 2.4 ± 0.5 | <0.0001* | 11.0 ± 1.8 | 0.0288* |
| High Dose | 1.2 ± 1.1 | 0.0004* | 0.4 ± 0.4 | 0.0013* | 2.3 ± 0.5 | <0.0001* | 9.0 ± 1.2 | <0.0001* |
| Prednisolone, | 3.8 ± 2.4 | 0.6134 | 0.9 ± 0.6 | 0.3777 | 4.0 ± 1.2 | 0.1538 | 13.3 ± 2.0 | 0.2819 |
| 10 mg/kg | | | | | | | | |

*p < 0.05 vs. vehicle
Number of inflammatory foci were compared using 2-tailed Student's t-test
Demyelination (anti-MBP) was compared using Wilcoxon's non-parametric test
Apoptosis (number of apoptotic cells) was compared using 2-tailed Student's t-test
lba1 and GFAP (% of total spinal cord area) compared using 2-tailed Student's t-test

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A pharmaceutical composition comprising, β-endorphin and adrenocorticotropic hormone (ACTH) or analogues thereof of either or both.

2. A pharmaceutical composition comprising, two active agents, wherein the active agents consist of β-endorphin and ACTH or analogues thereof.

3. The pharmaceutical composition of claim 1, which does not comprise any pro-opiomelanocortin (POMC)-derived peptides other than β-endorphin and ACTH.

4. A repository formulation comprising the pharmaceutical composition of claim 1.

5. The repository formulation of claim 4 comprising gelatin.

6. The repository formulation of claim 5 comprising 15-17% gelatin.

7. The composition of claim 1, wherein the β-endorphin has the sequence of human β-endorphin (SEQ ID NO: 1).

8. The composition of claim 1, wherein the β-endorphin has the sequence of bovine β-endorphin (SEQ ID NO: 3).

9. The composition of claim 1, wherein the β-endorphin has the sequence of porcine β-endorphin (SEQ ID NO: 5).

10. The composition of claim 1, wherein the ACTH has the sequence of human ACTH (SEQ ID NO: 2).

11. The composition of claim 1, wherein the ACTH has the sequence of bovine ACTH (SEQ ID NO: 4).

12. The composition of claim 1, wherein the ACTH has the sequence of porcine ACTH (SEQ ID NO: 6 or SEQ ID NO: 7).

13. A method of treating an inflammatory, autoimmune, allergic, or neurologic disease or disorder comprising administering the pharmaceutical composition of claim 1.

14. The method of claim 13, wherein the neurologic disorder is infantile spasm, Lennox-Gastaut syndrome, Landau-Kleffner syndrome, or electrical status epilepticus in sleep.

15. The method of claim 13, wherein the inflammatory or autoimmune disease is multiple sclerosis.

16. The method of claim 13, wherein the inflammatory or autoimmune disease is psoriatic arthritis; rheumatoid arthritis, ankylosing spondylitis, systemic lupus erythematosus or systemic dermatomyositis.

17. The method of claim 13, wherein the inflammatory or allergic disorder is an ophthalmic disease.

18. The method of claim 17, wherein the ophthalmic disease is keratitis, iritis, iridocyclitis, diffuse posterior uveitis and choroiditis, optic neuritis, chorioretinitis, or anterior segment inflammation.

19. The method of claim 13, wherein the inflammatory or allergic disorder is edema, severe erythema multiforme, Stevens-Johnson syndrome, serum sickness, or symptomatic sarcoidosis.

20. The method of claim 13, further comprising administration of Tempol.

21. A syringe, pre-filled with the formulation of claim 4.

22. (canceled)


SEQUENCE LISTING (11 sequences)

SEQ ID NO: 1 - protein, 31 aa, Homo sapiens
YGGFMTSEKSQTPLVTLFKNAIIKNAYKKGE

SEQ ID NO: 2 - protein, 39 aa, Homo sapiens
SYSMEHFRWGKPVGKKRRPVKVYPNGAEDESAEAFPLEF

SEQ ID NO: 3 - protein, 31 aa, Bos taurus
YGGFMTSEKSQTPLVTLFKNAIIKNAHKKGQ

SEQ ID NO: 4 - protein, 39 aa, Bos taurus
SYSMEHFRWGKPVGKKRRPVKVYPNGAEDESAQAFPLEF

SEQ ID NO: 5 - protein, 31 aa, Sus scrofa
YGGFMTSEKSQTPLVTLFKNAIVKNAHKKGQ

SEQ ID NO: 6 - protein, 39 aa, Sus scrofa
SYSMEHFRWGKPVGKKRRPVKVYPNGAEDELAEAFPLEF

SEQ ID NO: 7 - protein, 39 aa, Sus scrofa
SYSMEHFRWGKPVGKKRRPVKVYPDGAEDQLAEAFPLEF

SEQ ID NO: 8 - protein, 31 aa, Mus musculus; identical to SEQ ID NO: 3

SEQ ID NO: 9 - protein, 39 aa, Mus musculus
SYSMEHFRWGKPVGKKRRPVKVYPNVAENESAEAFPLEF

SEQ ID NO: 10 - protein, 266 aa, Homo sapiens
MPRSCCSRSGALLLALLLQASMEVRGWCLSSQCQDLTTESNLLECIRACKPDLSAETPMFPGNGDEQPLTENPRKYVMGHFRWDRFGRRNSSSSGSSGAGQKREDVSAGEDCGPLPEGGPEPRSDGAKPGPREGKRSYSMEHFRWGKPVGKKRRPVKVYPNGAEDESAEAFPLEFKRELTGQRLREGDGPDGPADDGAGAQADLEHSLLVAAEKKDEGPYRMEHFRWGSPPKDKRYGGFMTSEKSQTPLVTLFKNAIIKNAYKKGE

SEQ ID NO: 11 - protein, 130 aa, Homo sapiens
SYSMEHFRWGKPVGKKRRPVKVYPNGAEDESAEAFPLEFKRELTGQRLREGDGPDGPADDGAGAQADLEHSLLVAAEKKDEGPYRMEHFRWGSPPKDKRYGGFMTSEKSQTPLVTLFKNAIIKNAYKKGE